(19) United States
(12) Patent Application Publication
(10) Pub. No.: US 2016/0143628 A1
(43) Pub. Date: May 26, 2016

(54) ULTRASONIC DIAGNOSTIC APPARATUS

(71) Applicants: KABUSHIKI KAISHA TOSHIBA, Minato-ku (JP); Toshiba Medical Systems Corporation, Otawara-shi (JP)

(72) Inventors: Norihisa KIKUCHI, Otawara (JP); Kazutoshi SADAMITSU, Otawara (JP); Naoyuki NAKAZAWA, Otawara (JP); Fumiyasu SAKAGUCHI, Otawara (JP); Masatoshi NISHINO, Otawara (JP); Jiro HIGUCHI, Otawara (JP); Atsushi SUMI, Otawara (JP); Yutaka KOBAYASHI, Nasushiobara (JP); Cong YAO, Otawara (JP); Masami TAKAHASHI, Nasushiobara (JP); Atsushi NAKAI, Nasushiobara (JP)

(73) Assignees: KABUSHIKI KAISHA TOSHIBA, Minato-ku (JP); Toshiba Medical Systems Corporation, Otawara-shi (JP)

(21) Appl. No.: 14/947,300

(22) Filed: Nov. 20, 2015

(30) Foreign Application Priority Data
Nov. 21, 2014 (JP) 2014-236733

(51) Int. Cl. A61B 8/00; A61B 8/08

(52) U.S. Cl. CPC A61B 8/54; A61B 8/463; A61B 8/467; A61B 8/5207

(57) ABSTRACT

An ultrasonic diagnostic apparatus according to the embodiment includes a memory, a display, an input interface unit and a control unit. The memory stores a plurality of body marks that are classified for each region of a subject. The display displays the plurality of body marks stored in the memory. The input interface unit instructs selection of any one of the plurality of body marks displayed on the display. The control unit reads from the memory the plurality of body marks in the order from broader to narrower and displays the body marks on the display, in response to an input operation by means of the

### CROSS-REFERENCE TO RELATED APPLICATIONS

This application is based upon and claims the benefit of priority from the prior Japanese Patent Application No. 2014-236733, filed Nov. 21, 2014, the entire contents of which are incorporated herein by reference.

### FIELD

An embodiment described herein relates generally to, for example, a ultrasonic diagnostic apparatus that is capable of quickly and simply selecting a desired body mark by performing interactive operations for hierarchically classified items.

### BACKGROUND

Ultrasonic image diagnosis that applies ultrasonic waves to a subject and images a scanned region by using the reflected ultrasonic waves to enable a doctor to conduct a correct diagnosis is becoming widely popular. In ultrasonic diagnostic apparatuses for ultrasonic image diagnosis, body marks that illustrate examination target regions of a subject as drawings and annotations that indicate the status of the examination target regions as text have been used as an important means to explicitly retain the target regions for imaging in ultrasonic image diagnosis or the status of the target regions. Generally, it has been often carried out that selection candidates of body marks are displayed on a screen by the graphical user interface (GUI). However, it has been recently suggested and developed that the body marks are selected and displayed through a touch panel (or touch command screen (TCS)).

Ultrasonic diagnostic apparatuses usually require setting of optimal parameters (for example, ultrasonic wave frequency, focal distance, etc.) suitable for examination target regions or imaging methods. Conventional ultrasonic diagnostic apparatuses have presets of parameters for each examination target region to set a plurality of parameters at one time. Specifically, an item showing a name of an examination target region on a preset selection screen is pressed when an examination starts, or a name of an examination target region is selected when patient information is input. On the other hand, it has been suggested to display body marks on a touch command screen, and to change the display to show related examination regions (related image candidates) in response to a touch input operation of an operator (examiner) such as a doctor.

However, in the conventional technique to select a suitable mark from among a plurality of displayed body marks that are preset for each examination region, there is a need to search for a suitable body mark from multiple body marks. This increases the complexity of operation and inhibits quick and simple selection operations. Similarly, in the technique to display body marks on a touch command screen, and to change the display to show related examination regions in response to the touch input operations of an examiner, multiple related image candidates are displayed at once. Accordingly, this technique also involves the above mentioned operational complexity for the examiner.

### BRIEF DESCRIPTION OF THE DRAWINGS

FIG. 1 is a block diagram illustrating the configuration of an ultrasonic diagnostic apparatus of the embodiment.

FIG. 2A illustrates a hierarchy of multiple body marks to be selected.

FIG. 2B is a flowchart illustrating a detailed processing for a broad-to-narrow selection function of an ultrasonic diagnostic apparatus 1 according to one embodiment where an examiner uses the ultrasonic diagnostic apparatus 1.

FIG. 3A illustrates an aspect where body marks (first body marks) each indicating gender and a figure of a subject are displayed on a touch command screen.

FIG. 3B illustrates the examiner's touch input operation to select a desired body mark and an examination target region on the touch command screen in the aspect shown in FIG. 3A.

FIG. 4A illustrates an aspect where body marks (second body marks) indicating the selected examination target region are displayed on the touch command screen.

FIG. 4B illustrates the examiner's touch input operation to select a desired examination target organ on the touch command screen in the aspect shown in FIG. 4A.

FIG. 5A illustrates an aspect where a body mark (third body mark) of the selected examination target organ are displayed on the touch command screen.

FIG. 5B illustrates the examiner's operation of swiping or drawing a line on a desired portion of the examination target organ on the touch command screen in the aspect shown in FIG. 5A.

FIG. 5C illustrates an aspect where a probe mark is displayed on the touch command screen based on the operation shown in FIG. 5B.

FIG. 6 illustrates an example of a registration form including patient registration information presented on a display.

FIG. 7 illustrates an aspect where body marks of the examination target organ and the probe mark are displayed superimposed on a diagnostic image on the display.

### DETAILED DESCRIPTION

In general, according to one embodiment, the ultrasonic diagnostic apparatus includes a memory, a display, an input interface unit, and a control unit. The memory stores a plurality of body marks that are classified for each region of a subject. The display displays the plurality of body marks stored in the memory. The input interface unit instructs selection of any one of the plurality of body marks displayed on the display. The control unit reads from the memory the plurality of body marks from a broader region to a narrower region of the subject and displays the body marks on the display, in response to an input operation to the input interface unit.

Hereinafter, embodiments will be described with reference to the drawings. In the description below, structural elements having substantially the same configurations will be denoted by the same reference symbols, and a repetitive description will be given only where necessary.

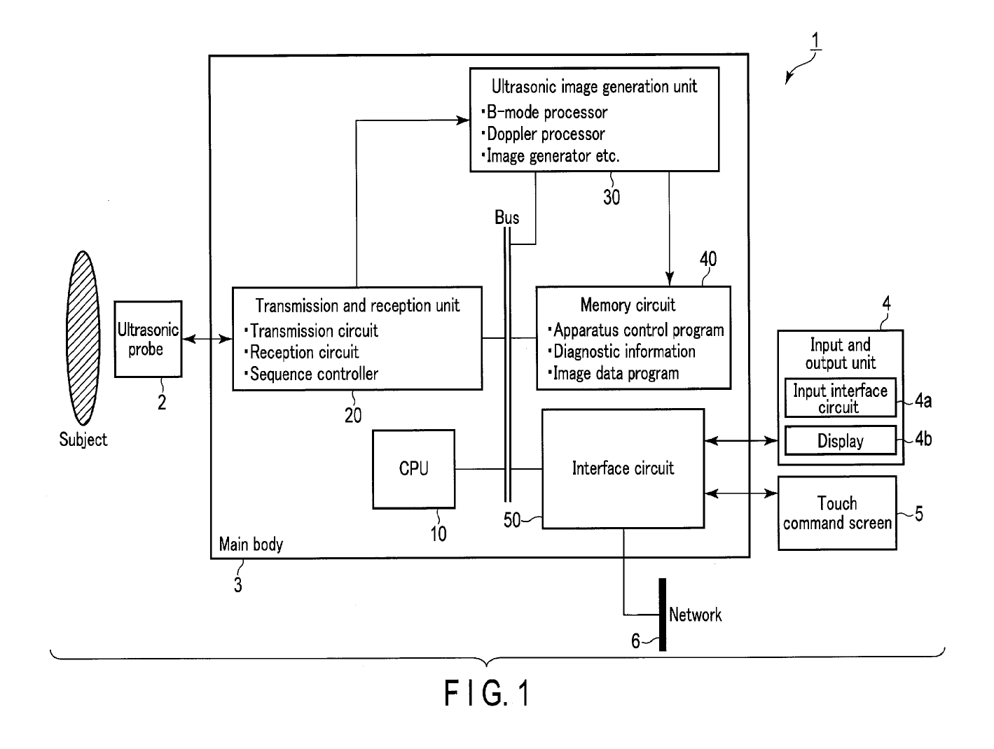

FIG. 1 is a block diagram illustrating the configuration of an ultrasonic diagnostic apparatus 1 of the embodiment. The ultrasonic diagnostic apparatus 1 includes an ultrasonic probe 2, a main body 3, an input and output unit 4, and a touch command screen 5 (TCS). The ultrasonic diagnostic apparatus 1 may be connected to a network 6 capable of communicating with an external device through an interface circuit 50 of the main body 3.

The ultrasonic probe 2 includes a plurality of piezoelectric vibrators, a matching layer, and backing materials provided on the back side of the piezoelectric vibrators. The plurality of piezoelectric vibrators are acoustic/electric reversible sensing elements such as piezoelectric ceramics. The plurality of piezoelectric vibrators are arranged in parallel with each other and are provided at the distal end of the ultrasonic probe 2. The piezoelectric vibrators generates ultrasonic waves in response to a driving pulse transmitted and supplied from a transmission circuit in the transmission and reception unit 20.

When ultrasonic waves are applied to the subject through the ultrasonic probe 2, the applied ultrasonic waves are reflected on the surface where acoustic impedance is discontinuous in a living tissue of the subject. The piezoelectric vibrators each receive the reflected ultrasonic waves and generate an echo signal. The reception circuit in the transmission and reception unit 20, when receiving the echo signal, transmits a receive signal that is based on the echo signal to a ultrasonic image generation unit 30 described below.

The amplitude of the echo signal depends on the difference in the acoustic impedance at the boundary showing discontinuity of the acoustic impedance that affects the reflection of ultrasonic waves. The piezoelectric vibrator for generating ultrasonic waves (for transmission) and the piezoelectric vibrator for generating echo signals (for reception) may be the same piezoelectric vibrator by setting the timings of transmission and reception if discrete ultrasonic waves (pulse waves) are applied, for example. Otherwise, the piezoelectric vibrator for transmission and the piezoelectric vibrator for reception may be independently provided if, for example, continuous ultrasonic waves (continuous waves) are applied. The frequency of an echo signal reflected on a bloodstream through which transmission ultrasonic waves move, or on a surface such as the cardiac wall, is shifted depending on velocity components in the direction of the transmitted ultrasonic waves of a moving object (bloodstream and surface of the cardiac wall) due to the Doppler effect.

The main body 3 includes a central processing unit (CPU) 10, the transmission and reception unit 20, the ultrasonic image generation unit 30, a memory circuit 40, and the interface circuit 50.

The CPU 10 (processing circuit/control circuit) mainly controls the entire operation of the ultrasonic diagnostic apparatus 1. The control of the CPU 10 will be explained in detail in relation to the other units.

The transmission and reception unit 20 includes the transmission circuit, the reception circuit, and a sequence controller, for example.

The transmission circuit transmits a driving pulse to each of the plurality of piezoelectric vibrators in the ultrasonic probe 2, as stated above.

The reception circuit generates a receive signal based on an echo signal generated by each piezoelectric vibrator, and transmits the receive signal to the ultrasonic image generation unit 30, as stated above.

The sequence controller controls a transmission sequence of the driving pulse for generating ultrasonic waves with a high time resolution (for example, on the order of several hundred milliseconds).

The ultrasonic image generation unit 30 includes a B-mode processor, a Doppler processor, and an image generator, for example.

The B-mode processor includes an envelope detection circuit, and a logarithmic transformation circuit, for example. The envelope detection circuit performs envelope detection to the receive signal output from the reception circuit. The envelope detection circuit outputs the signal which has been subjected to the envelope detection to the logarithmic transformation circuit. The logarithmic transformation circuit relatively enhances a weak signal by performing logarithmic transformation to the signal which has been subjected to the envelope detection. The B-mode processor generates a signal value for the depth of ultrasonic transmission and reception for each scanning line and based on the enhanced signal by the logarithmic transformation circuit. The B-mode processor may generate volume data instead of the signal value for the depth of ultrasonic transmission and reception for each scanning line. Hereinafter, data generated at the B-mode processor is called B-mode data.

The Doppler processor includes a mixer, a lowpass filter (LPF), and a velocity/dispersion/power arithmetic circuit, for example. The mixer multiplies the receive signal output from the reception circuit by a reference signal having a frequency f₀ that is the same as the transmission frequency. By this multiplication, a signal having a component of Doppler shift frequency f_{d} and a signal having a frequency component of (2f₀+f_{d})is obtainable. The LPF removes a signal having a higher frequency component (2f₀+f_{d}) among signals having two kinds of frequency components received from the mixer. By removing the signal having the higher frequency component (2f₀+f_{d}), a Doppler signal having the component of Doppler shift frequency f_{d} is generated.

The Doppler processor may use a quadrature detection method to generate a Doppler signal. In this case, the receive signal is subjected to quadrature detection, and converted to an IQ signal. The Doppler processor generates a Doppler signal having the component of Doppler shift frequency f_{d} by performing a complex Fourier transform to the IQ signal. The Doppler signal is a Doppler component defined by a bloodstream, tissue, or contrast agent, for example. The velocity/dispersion/power arithmetic circuit includes a moving target indicator (MTI) filter, the LPF, and an autocorrelation arithmetic circuit, for example. The velocity/dispersion/power arithmetic circuit may include a cross-correlation arithmetic circuit instead of the autocorrelation arithmetic circuit. The MTI filter removes a Doppler component (clutter component) caused by respiratory movement or pulsatile movement of organs from the generated Doppler signal. The MTI filter is used to extract a Doppler component relating to a bloodstream from the Doppler signal. The LPF is used to extract a Doppler component relating to movement of tissues from the Doppler signal.

The image generator includes a digital scan converter (DSC) and an image memory. The image generator performs coordinate conversion processing (re-sampling) to the DSC. The coordinate conversion processing converts a scanning line signal array of an ultrasonic scan, for example the B-mode data, Doppler data, and propagation time data, into a scanning line signal array of the general video format such as a format used for television. The image generator generates an ultrasonic image as a display image by the coordinate conversion processing. Specifically, the image generator generates a B-mode image based on the B-mode data. The image generator generates a Doppler image such as an average velocity image, a dispersion image, and a power image, based on the Doppler data. A superimposed image in which text information (annotations) of various parameters and scales are superimposed on the ultrasonic image may be generated.

The memory circuit 40 stores various data group such as transmission/reception conditions, a diagnostic protocol, a control program of the ultrasonic diagnostic apparatus 1, diagnostic information (patient ID, doctor's observation, etc.), the receive signal generated by the reception circuit, B-mode data generated by the B-mode processor, Doppler data generated by the Doppler processor, the B-mode image, the average velocity image, and the dispersion image. The memory circuit 40 includes a memory not shown in the drawings, and stores data (image data) corresponding to the generated ultrasonic image (B-mode image, average velocity image, dispersion image, power image, etc.). The image data stored in the memory is read in response to an instruction of the examiner through an input interface circuit 4a of the input and output unit 4 described later. The memory is a cine-memory that stores, for example, an ultrasonic image corresponding to a plurality of frames immediately before freezing. Displaying images stored in the cine-memory continuously (cine display) may allow the ultrasonic image to be displayed on a display 4b of the input and output unit 4. The CPU 10 executes a program stored in the memory circuit 40, for example. The CPU 10 performs writing and reading of the data with respect to the memory.

The memory circuit 40 also stores supplemental information attached to the ultrasonic image and the history of using the supplemental information. The supplemental information includes a body mark (first body mark) and a probe mark (second body mark) placed on the body mark. The body mark is a drawing that illustrates a body of a subject to indicate which part of the body is to be diagnosed. In the ultrasonic diagnostic apparatus 1 according to the embodiment, the memory circuit 40 stores a plurality of body marks illustrating at least a region of the subject, the body marks being classified into levels of the anatomical hierarchy (category, hierarchy, or broad/narrow (detail)).

For instance, the memory circuit 40 stores body marks indicating types of the subject (at least one of gender, body shape, whether the subject is an adult, or is pregnant), body marks indicating examination target regions (abdomen, chest, etc.), and body marks indicating examination target organs (heart, liver, etc.). Body marks indicating types of the subject used to select an examination target region are referred to as first body marks, body marks indicating the selected examination target region used to select an examination target organ are referred to as second body marks, and a body mark that is a close-up image of the selected examination target organ is referred to as a third body mark.

The probe mark is a drawing placed on a body mark and illustrating a probe shape to indicate an organ or a place of an organ of the subject to be imaged. The supplemental information includes information regarding where in the body mark the probe mark is placed, and at which angle the probe mark is placed.

The memory circuit 40, described hereinafter, stores various programs to display the body marks and probe marks on the touch command screen 5.

The memory circuit 40 may store annotations other than the body marks and probe marks. The annotations are text indicating information concerning examination target regions (for example, notes regarding a symptom of the examination target regions), and are input on the ultrasonic image. The supplemental information includes information regarding what kind of text is input as an annotation, and where in the ultrasonic image the annotation is input.

The supplemental information such as the body marks, probe marks, and annotations are used not only for examination, but also for checking various information after the examination.

The interface circuit 50 is an interface relating to the input and output unit 4, the touch command screen 5, and the network 6. The analysis results and the data such as ultrasonic image obtained by the main body 3 may be transferred to another device through the interface circuit 50 and the network 6. The interface circuit 50 may download a medical image regarding the subject obtained by another medical image diagnosis apparatus through the network 6.

The input and output unit 4 includes an input interface circuit 4a such as a track ball, a switch button, a mouse, and a keyboard, and a display 4b such as a CRP monitor and a liquid crystal monitor.

The input interface circuit 4a is connected to the interface circuit 50, and inputs various instructions (order, information, selection, setting, etc.) from the examiner to the main body 3. In particular, the text stored as an annotation is transmitted to the CPU 10 through a keyboard, for example. The annotation may be transmitted to the CPU 10 by inputting any characters using a keyboard, or by selecting any character strings from a predefined set of character strings.

The display 4b mainly displays the ultrasonic image or the superimposed image generated at the image generator.

The touch command screen 5 is an electronic part including a display device such as a liquid crystal panel and a position input device such as a touch pad. That is, the touch command screen 5 is a display that accepts a touch input operation (contact input instruction) by the examiner. A plurality of buttons each indicating a candidate of supplemental information to be attached to the ultrasonic image is displayed on the touch command screen 5. The examiner may input the supplemental information to be attached to the ultrasonic image by touching a desired button. The broad-to-narrow selection function (or hierarchical body mark selection function) of the ultrasonic diagnostic apparatus 1 according to the embodiment is executed through the touch command screen 5.

The touch command screen 5 of the ultrasonic diagnostic apparatus 1 according to the embodiment is an example, and is not limited thereto. For example, the function of the touch command screen 5 may be executed by using any devices having an input function (track ball, switch button, mouse, keyboard, etc.), and any devices having a display function (liquid crystal monitor, CRT monitor, etc.).

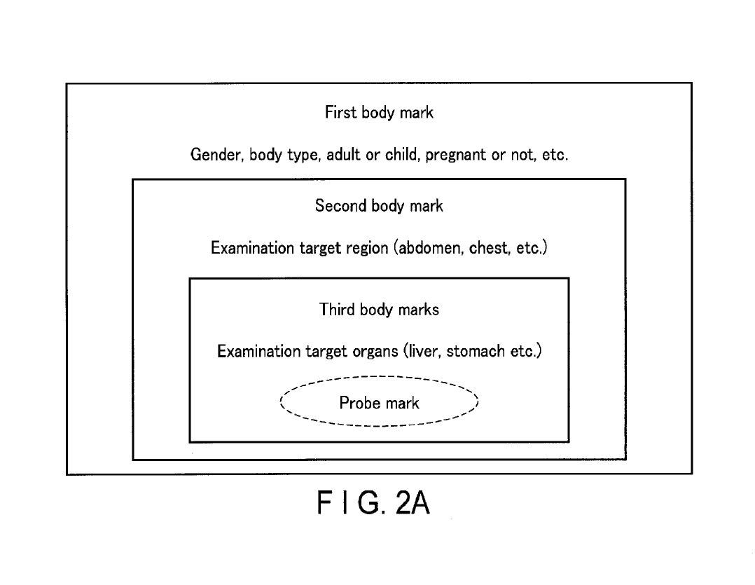

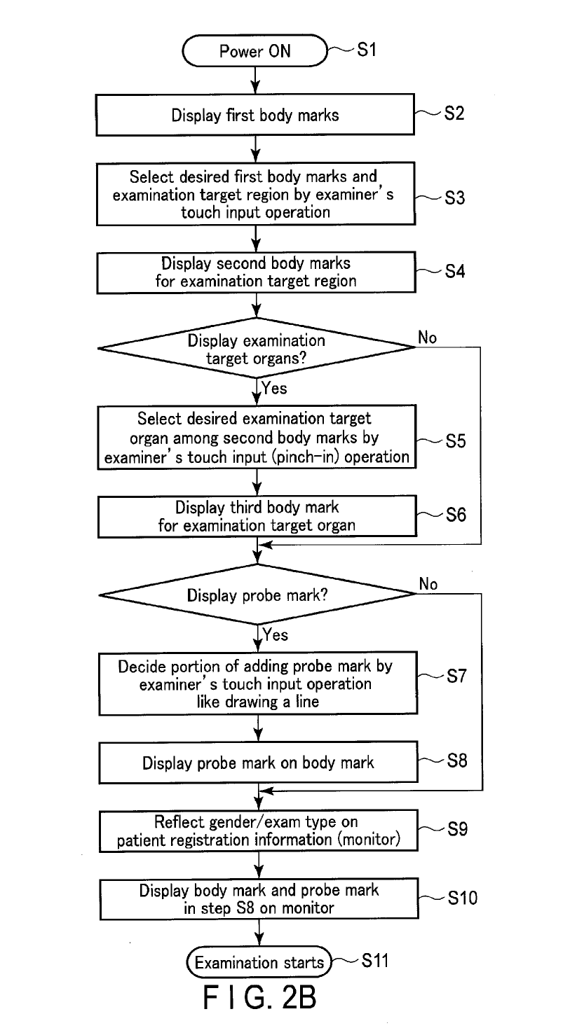

FIG. 2A illustrates a hierarchy of multiple body marks to be selected. The ultrasonic diagnostic apparatus 1 displays the body marks on the touch command screen 5 by switching the levels of the hierarchy. FIG. 2A illustrates body marks having a three-level hierarchy and a probe mark (if necessary) that is displayed after the selection of a body mark in the third level of hierarchy A modified example having a two-level hierarchy or a hierarchy of four or more levels may be embodied.

The examiner may select a desired body mark and set a probe mark in each level of the hierarchy. For example, in the first level of the hierarchy (e.g., broadest level), the examiner may select a desired type of subject and a desired examination target region from the first body marks indicating the types of subjects that are displayed on the touch command screen 5. In the second level of the hierarchy (e.g., middle level), the examiner may select a desired examination target organ from the second body marks indicating the examination target region that is selected in the first level and displayed on the touch command screen 5. In the third level of the hierarchy (e.g., narrowest level), the examiner may set a probe mark on a desired position on the third body mark that is a close-up image of the selected examination target organ. The above-mentioned selection function is referred to as a “broad-to-narrow selection function”. The details about each level of the hierarchy will be explained below.

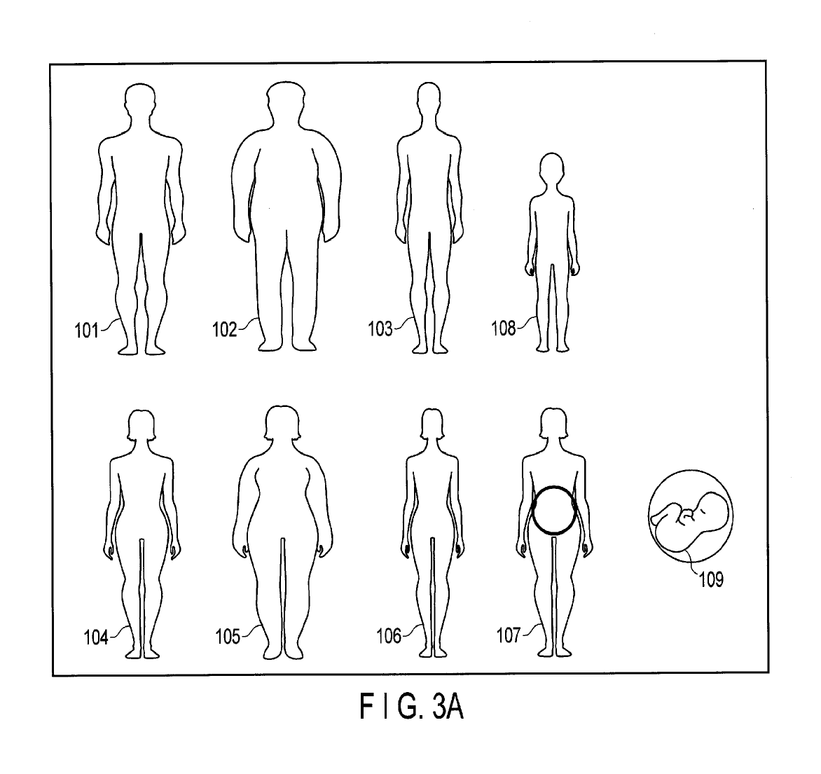

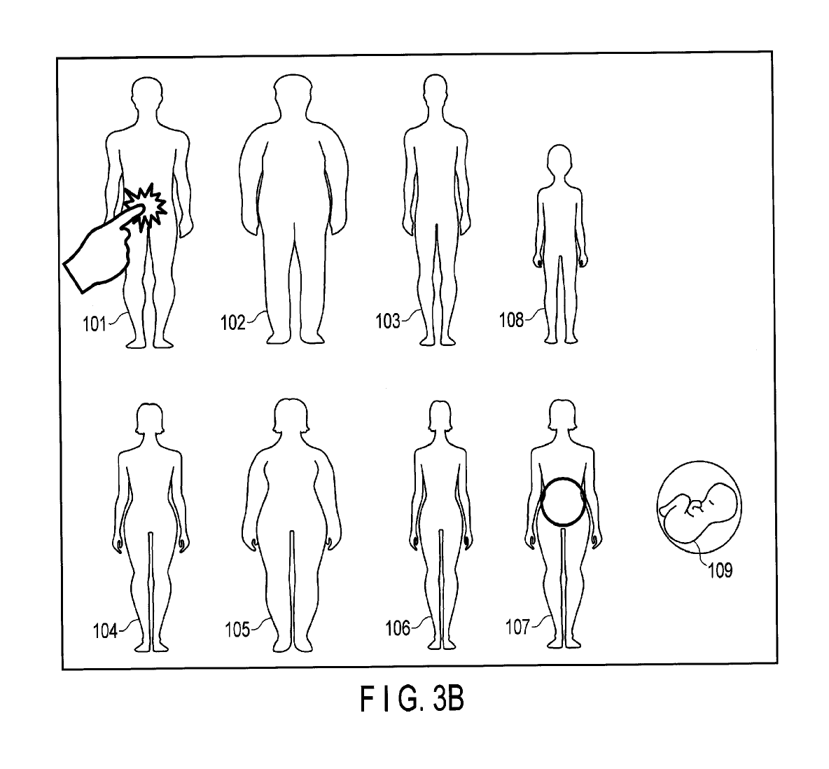

FIG. 2B is a flowchart illustrating a detailed processing for a broad-to-narrow selection function of an ultrasonic diagnostic apparatus 1 according to the embodiment where the examiner uses the ultrasonic diagnostic apparatus 1. The embodiment will be explained with reference to each step shown in FIG. 2B, and FIGS. 3-7 respectively corresponding to the steps of FIG. 2B.

[Step S1]

The ultrasonic diagnostic apparatus 1 is powered on by the examiner's operation of turning the power supply switch to ON.

[Step S2]

FIG. 3A illustrates an aspect (first level) where body marks (first body marks 100) each indicating types of subjects are displayed on a touch command screen.

The CPU 10 executes a predetermined program with regard to the supplemental information stored in the memory circuit 40 to display the first body marks 100 (whole-body marks) indicating the types of subjects on the touch command screen 5. In the embodiment shown in FIG. 3, the touch command screen 5 displays a body mark 101 (standard male body type), a body mark 102 (endomorph male), a body mark 103 (ectomorph male), a body mark 104 (standard female body type), a body mark 105 (endomorph female), a body mark 106 (ectomorph female), a body mark 107 (expectant mother), a body mark 108 (child), and a body mark 109 (fetus).

[Step S3]

FIG. 3B illustrates the examiner's touch input operation to select a desired body mark and an examination target region on the touch command screen 5 in the aspect shown in FIG. 3A.

As show in FIG. 3B, the examiner performs a touch input operation (first input operation) to select a desired body mark (for example, body mark 101 (standard male body type)) among the first body marks 100 displayed on the touch command screen 5 in step S2, and to select an examination target region (for example, the abdomen). In FIG. 3B, the abdomen of the standard male body type is selected.

[Step S4]

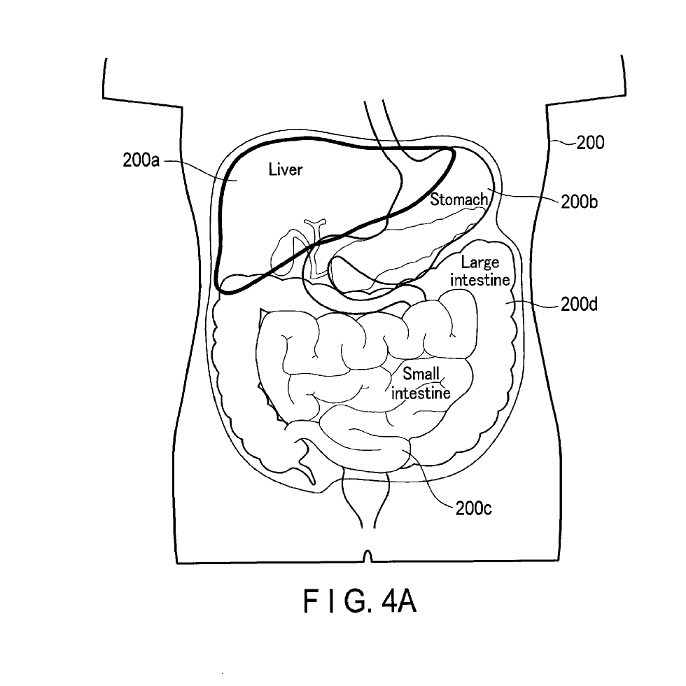

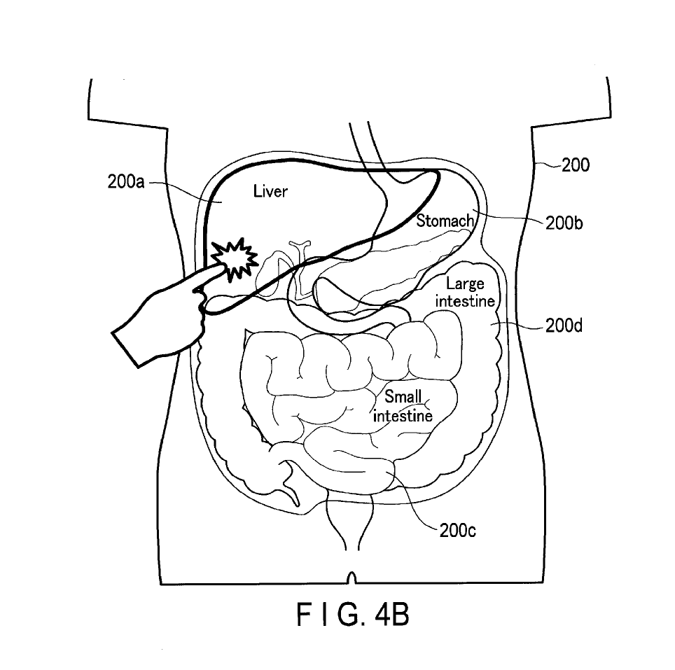

FIG. 4A illustrates an aspect (second level) where body marks (second body marks 200) each indicating an examination region are displayed on the touch command screen.

In response to the touch input operation in step S3, the touch command screen 5 transmits an input signal to the CPU 10. The CPU 10 executes a predetermined program with regard to the supplemental information stored in the memory circuit 40 to display the second body marks 200 indicating the selected examination region and placed on the level next to the first body marks 100. Since the abdomen of the standard male body type has been selected on the first level, the second body marks 200 indicate the details of the abdomen that include a liver 200a, stomach 200b, small intestine 200c, and large intestine 200d.

[Step S5]

FIG. 4B illustrates the examiner's touch input operation to select a desired examination target organ on the touch command screen 5 in the aspect shown in FIG. 4A.

As shown in FIG. 4B, the examiner performs a touch input operation to select a desired examination target organ (for example, liver 200a) among the second body marks 200 displayed on the touch command screen 5 in step S4. In

FIG. 4B, the liver 200a is selected. The desired examination target organ may be selected by a pinch-in operation (squeezing an object between two fingers).

[Step S6]

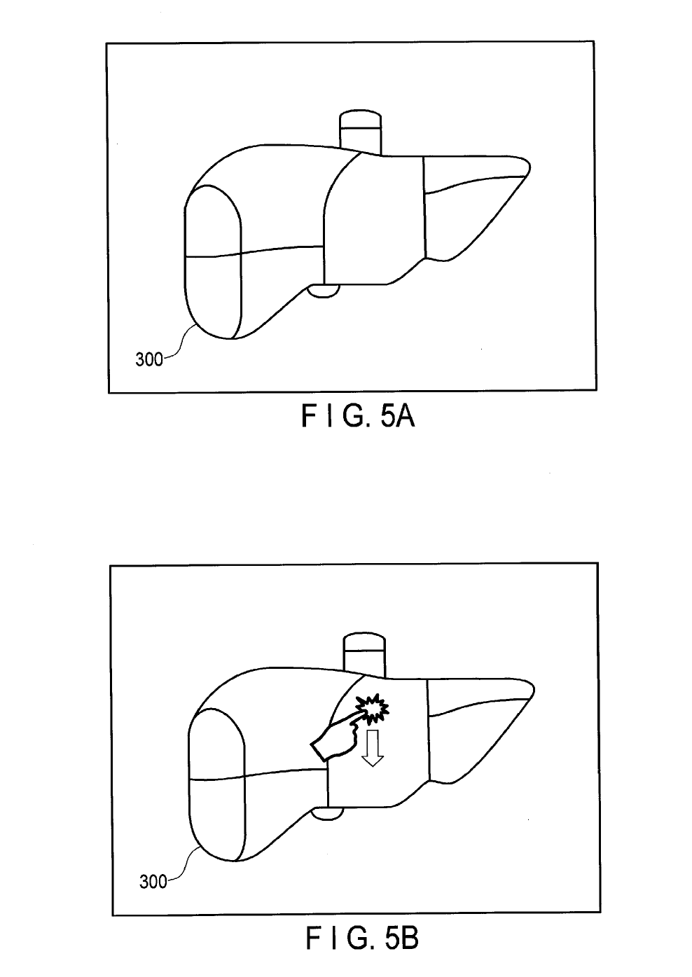

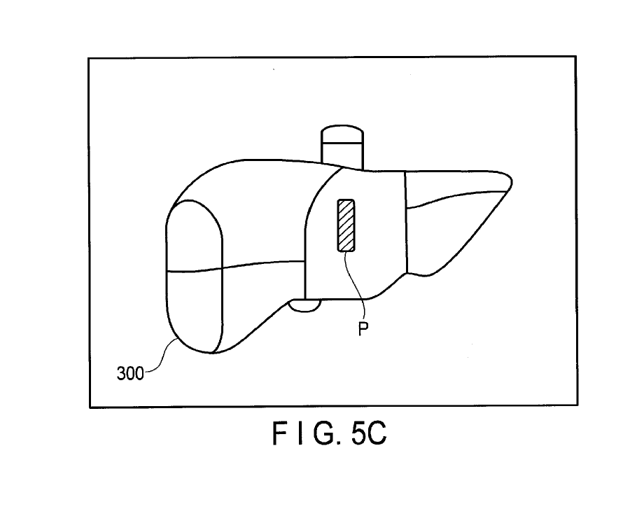

FIG. 5A illustrates an aspect (third level) where a body mark (third body mark 300) of the selected examination target organ is displayed on the touch command screen.

In response to the touch input operation (or pinch-in operation) in step S5, the touch command screen 5 transmits an input signal to the CPU 10. The CPU 10 executes a predetermined program with regard to the supplemental information stored in the memory circuit 40 to display the third body mark 300 indicating the selected examination target organ and placed on the level next to the second body marks 200. Since the liver 200a in the abdomen is selected on the second level, the third body mark 300 that is a close-up image of the liver 200a is displayed.

In the actual examination, there may be a case where the examiner wishes to place a probe mark described later on the selected examination target region, instead of on the selected organ (i.e., placing a probe mark in the state where the second body marks 200 indicating the abdomen are displayed in this embodiment). In such a case, steps S5 and S6 may be skipped.

[Step S7]

FIG. 5B illustrates the examiner's operation of swiping or drawing a line (second input operation) on a desired portion of the selected examination target organ on the touch command screen in the aspect shown in FIG. 5A.

As shown in FIG. 5B, the examiner touches a portion where a probe mark P is to be displayed of the third body marks 300 (second body marks 200 if steps S5 and S6 are skipped) displayed on the touch command screen 5 in the step S6, and swipes the portion like drawing a line.

[Step S8]

FIG. 5C illustrates an aspect where a probe mark P is displayed on the touch command screen based on the operation shown in FIG. 5B.

In response to the input operation in step S7, the touch command screen 5 transmits an input signal to the CPU 10. The CPU 10 executes a predetermined program with regard to the supplemental information stored in the memory circuit 40 to superimpose the probe mark P on the third body marks 300 displayed on the touch command screen 5.

If a probe mark P is not necessary to be displayed, steps S7 and S8 may be skipped.

[Step S9]

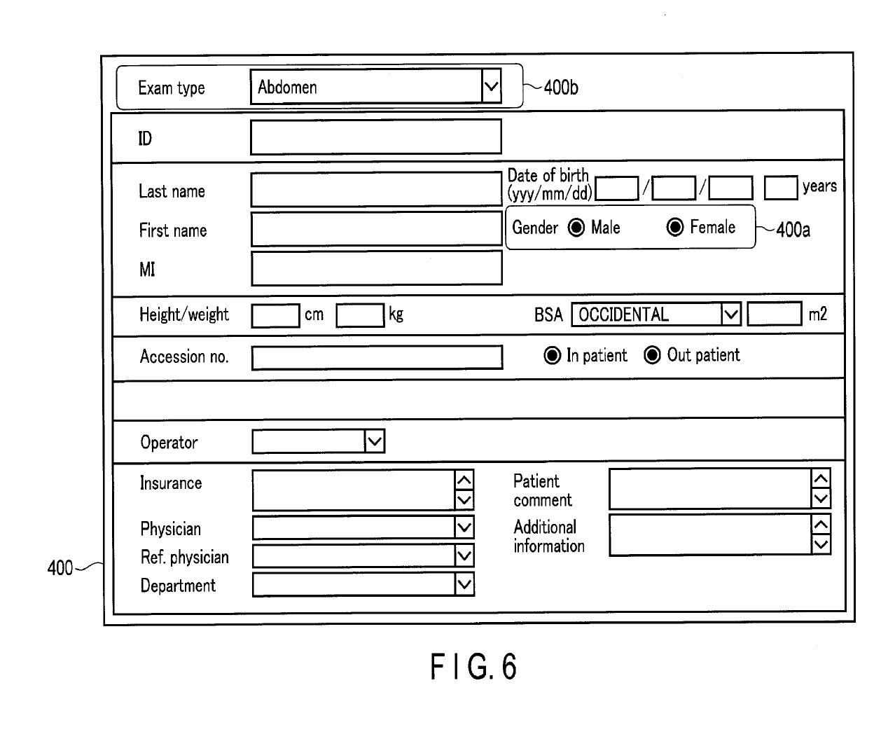

FIG. 6 illustrates an example of a registration form 400 including patient registration information presented on the display 4b.

The CPU 10 executes a predetermined program with regard to the supplemental information stored in the memory circuit 40 to generate patient registration information based on the type of subject, the examination target region, and the examination target organ input by the examiner in steps S3 and S5. The registration form 400 for the patient registration information displayed on the display 4b (item 400a, “gender” and item 400b, “exam type” shown in FIG. 6) is updated (or reflected) based on the type of subject, the examination target region, and the examination target organ input by the examiner in steps S3 and S5.

[Step S10]

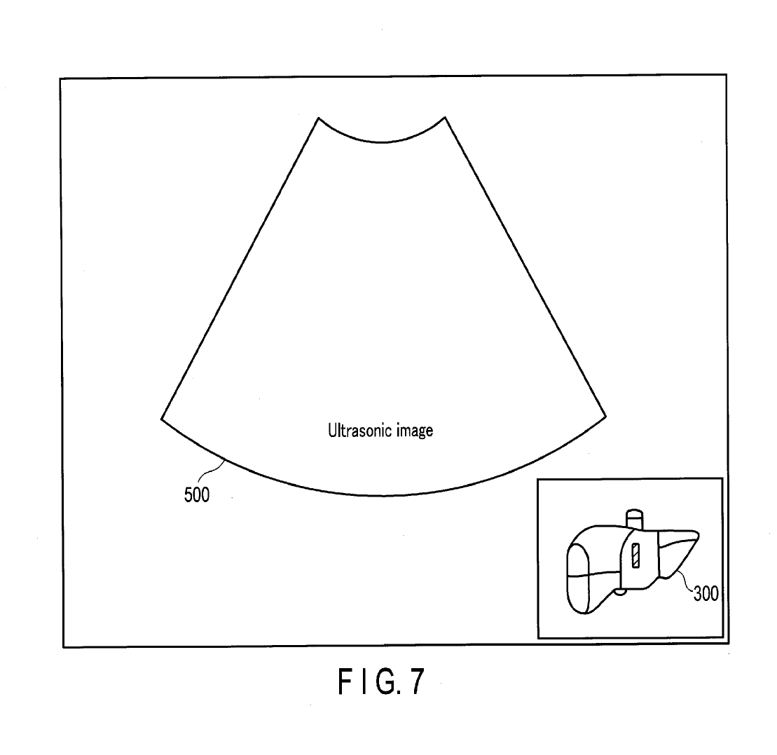

FIG. 7 illustrates an aspect where the display window of the touch command screen in step S8 (the image that the probe mark is superimposed on the third body marks 300) is displayed together with a diagnostic image on the display 4b.

Based on the patient registration information updated in step S9, the CPU 10 executes a predetermined program with regard to the supplemental information stored in the memory circuit 40 to display the third body mark 300 selected in the step S5 and displayed in step S6 (and the probe mark P input in step S7 and displayed in step S8 if steps S7 and S8 are performed) on the display 4b of the input and output unit 4, for example in the lower-right corner of the display 4b.

[Step S11]

The examination starts.

The ultrasonic diagnostic apparatus 1 according to the embodiment realizes the following advantages:

The ultrasonic diagnostic apparatus 1 has the above-mentioned broad-to-narrow selection function. That is, the body marks are displayed by switching the levels of hierarchy from the anatomically broader level to the anatomically narrower level on the touch command screen 5. In other words, the body marks are displayed in the broader to narrower order for the region of the subject. Accordingly, the examiner can set optimal parameters suitable for the examination target regions. In particular, in comparison with the conventional technique, there is no need of searching for a suitable switch from among a number of exam type switches indicating examination target regions, thereby achieving quick, easy, correct, and instinctive selection of examination target regions. In addition, the body marks in each level of the hierarchy are displayed by switching the levels, not at the same time. This achieves quick, easy, correct and instinctive selection of examination target regions in comparison with the conventional technique of displaying multiple candidate images. Furthermore, the information selected by the above-mentioned operation can be immediately reflected on the registration form 400 for the patient registration information displayed on the display 4b.

[Modifications]

The ultrasonic diagnostic apparatus 1 according to the embodiment has the above-mentioned broad-to-narrow selection function for the body marks. However, the broad-to-narrow selection function is not limited to the selection of body mark display. The identification information regarding regions of the subject may be selected in the order from broader to narrower (hierarchically) through the touch command screen 5, for example. In such a case, conditions (imaging and displaying conditions) regarding imaging and/or displaying corresponding to the identification information for the regions are set. The conditions regarding imaging and/or displaying may include the frequency of ultrasonic waves to be transmitted, and the depth from the body surface to the examination target region, for example. In other words, since the frequency suitable for each region and the depth of each region varies, the broad-to-narrow selection function is used as a tool for setting the conditions regarding imaging and/or displaying. In addition, the examiner has to select a suitable ultrasonic probe depending on an examination target region or an imaging mode when performing the ultrasonic diagnosis. Accordingly, it is effective to select a suitable ultrasonic probe by using the broad-to-narrow selection function through the touch command screen 5 by associating the probes with the identification information.

Note that a plurality of units or apparatuses according to the present embodiment may be implemented by processors or processing circuitry. The processing circuitry may be constituted of a singular set of circuitry such as a CPU, plural sets of circuitry corresponding to each of the units, or the combination thereof.

Furthermore, the word “processor” or “processing circuitry” used in the above description means circuitry such as a CPU (Central Processing Unit), a GPU (Graphics Processing Unit), an ASIC (Application Specific Integrated Circuit), a programmable logic device (e.g., an SPLD (Simple Programmable Logic Device), a CPLD (Complex Programmable Logic Device), or an FPGA (Field Programmable Gate Array)), or the like. The processor implements functions by reading out programs stored in the storage circuit and executing the programs. Note that it is possible to directly incorporate programs in the circuit of the processor instead of storing the programs in the storage circuit. In this case, the processor implements functions by reading out programs incorporated in the circuit and executing the programs.

Note that each processor in each embodiment described above may be formed as one processor by combining a plurality of independent circuits to implement functions as well as being formed as a single circuit for each processor. In addition, a plurality of constituent elements in each embodiment described above may be integrated into one processor to implement its function.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus comprising:
a memory that stores a plurality of body marks that are classified for each region of a subject in an order from broader to narrower;
a display that displays the plurality of body marks stored in the memory;
an input interface unit implemented by circuitry, the input interface unit instructing selection of any one of the plurality of body marks displayed on the display; and
a control unit implemented by circuitry, the control unit reading from the memory the plurality of body marks in the order from broader to narrower and displaying the body marks on the display, in response to an input operation by means of the input interface unit.

2. The ultrasonic diagnostic apparatus according to claim 1, wherein the memory stores the plurality of body marks that are classified into anatomical hierarchical levels.

3. The ultrasonic diagnostic apparatus according to claim 2, wherein the control unit allows the plurality of body marks to be displayed on the display in an order from broader to narrower in an anatomical hierarchy.

4. The ultrasonic diagnostic apparatus according to claim 1, wherein the control unit reads a body mark selected by the input operation by means of the input interface unit, and allows the selected body mark to be displayed on the display.

5. The ultrasonic diagnostic apparatus according to claim 1, wherein the memory stores a probe mark indicative of an imaging position; and
the control unit reads the probe mark from the memory, and superimposes the probe mark on a predetermined position of a screen of the display that has been selected by the input operation by means of the input interface unit.

6. The ultrasonic diagnostic apparatus according to claim 1, wherein the input interface unit is a touch panel having a display function and an input function.

7. The ultrasonic diagnostic apparatus according to claim 6, wherein the control unit reads a body mark in response to the input operation to a predetermined body mark displayed on the touch panel, and switches the display on the touch panel to the read body mark.

8. The ultrasonic diagnostic apparatus according to claim 6, wherein the memory stores a probe mark indicative of an imaging position; and
the control unit reads the probe mark from the memory and superimposes the probe mark on a position where the input operation has been entered on the touch panel.

9. The ultrasonic diagnostic apparatus according to claim 1, wherein the plurality of body marks include a first body mark illustrating a type of subject.

10. The ultrasonic diagnostic apparatus according to claim 9, wherein the type of subject includes at least one of gender, body type, and whether the subject is an adult, or is pregnant.

11. The ultrasonic diagnostic apparatus according to claim 1, wherein the plurality of body marks include a second body mark illustrating an examination target region.

12. The ultrasonic diagnostic apparatus according to claim 1, wherein the plurality of body marks include a third body mark illustrating an examination target organ.

13. The ultrasonic diagnostic apparatus according to claim 1, wherein the control unit generates patient registration information based on at least one body mark selected by the input operation by means of the input interface unit.

14. An ultrasonic diagnostic apparatus comprising:
a memory that stores a plurality of items of identification information that are classified for each region of a subject in an order from broader to narrower, and an imaging and displaying condition regarding at least one of imaging and displaying in accordance with the identification information;
a display that displays the plurality of items of identification information stored in the memory;
an input interface unit implemented by circuitry, the input interface unit instructing selection of any one of the plurality of items of identification information displayed on the display; and
a control unit implemented by circuitry, the control unit reading from the memory the plurality of items of identification information and the imaging and displaying condition according to the identification information in the order from broader to narrower and controlling at least one of imaging and displaying based on the imaging and displaying condition, in response to an input operation by means of the input interface unit.

15. The ultrasonic diagnostic apparatus according to claim 14, wherein the memory stores a depth from a body surface to an examination target region of the subject, a frequency of ultrasonic waves to be transmitted, and a probe to be used as the imaging and displaying condition.
